(19) United States
(12) Patent Application Publication
(10) Pub. No.: US 2026/0207065 A1
(43) Pub. Date: Jul. 23, 2026

(54) Blood Pressure Measuring Device and Method

(71) Applicant: KOROT CO., Ltd., Seoul (KR)

(72) Inventors: Ki Chul Cha, Seoul (KR); Jong Heun Shin, Seoul (KR); Sang Jin Lee, Suwon-si (KR)

(73) Assignee: KOROT CO., Ltd., Seoul (KR)

(21) Appl. No.: 19/162,053

(22) PCT Filed: Mar. 6, 2024
(86) PCT No.: PCT/KR2024/002827

§ 371 Date: Sep. 3, 2025

(30) Foreign Application Priority Data
Mar. 14, 2023 (KR) 10-2023-0033328
Mar. 4, 2024 (KR) 10-2024-0030836

(51) Int. Cl. A61B 5/022; A61B 5/00

(52) U.S. Cl. CPC A61B 5/02208; A61B 5/02225; A61B 5/02233; A61B 5/7264; A61B 5/742

(57) ABSTRACT

A blood pressure measuring device and method are disclosed. The disclosed blood pressure measuring device comprises a processor that displays signals sensed from a subject in real time on a display while the internal pressure of a cuff wrapped around a portion of the subject's body varies, and that visually varies a reference signal or displays a visual mark indicative of the reference signal when displaying the reference signal corresponding to the subject's blood pressure from among the signals on the display in real time.

### CROSS-REFERENCE TO RELATED APPLICATIONS

The current application is a national stage of PCT Application No. PCT/KR 2024/002827 filed Mar. 6, 2024, which claims priority to Korean Patent Application No. 10-2023-0033328 filed Mar. 14, 2023 and Korean Patent Application No. 10-2024-0030836 filed Mar. 4, 2024, the disclosures of which are hereby incorporated by reference in their entireties for all purposes.

### TECHNICAL FIELD

The following description relates to a blood pressure measuring device and method.

### TECHNICAL FIELD

The following description relates to a blood pressure measuring device and method.

### BACKGROUND ART

There are two types of blood pressure measuring methods: invasive and non-invasive methods. The invasive method may be the most accurate, but the non-invasive method is mainly used for reasons such as pain or infection. The non-invasive method may include auscultation and oscillometry, both of which use a cuff. Auscultation is a method of listening, through a stethoscope, to sound energy generated by turbulence during the movement of blood flow due to the opening and closing of blood vessels. Oscillometry is mainly used in blood pressure monitors and may have lower accuracy than auscultation.

### DISCLOSURE OF THE INVENTION

### Technical Goals

The present disclosure may provide a blood pressure measuring device and method that combine the advantages of auscultation and oscillometry by using the characteristics (e.g., a correlation between Korotkoff sounds and oscillometric signals, etc.) of Korotkoff sounds and oscillometric signals.

However, the technical aspects are not limited to the aforementioned aspects, and other technical aspects may be present.

### Technical Solutions

A blood pressure measuring device according to an embodiment includes a processor configured to display signals sensed from a subject in real time on a display while an internal pressure of a cuff wrapped around a portion of a body of the subject varies, and visually vary a reference signal or display a visual mark indicative of the reference signal when the reference signal corresponding to a blood pressure of the subject among the signals is displayed in real time on the display.

The processor may display the signals on the display based on a Korotkoff sound included in a sound signal collected from a microphone while the internal pressure of the cuff varies.

The processor may, when a signal that is not sensed from the subject is sensed while the internal pressure of the cuff varies, select the signal as a candidate reference signal, and determine whether to determine the candidate reference signal to be the reference signal based on whether a new signal is sensed from the subject after the candidate reference signal.

The processor may determine the candidate reference signal to be the reference signal in response to n (where n is a natural number) new signals being continuously sensed from the subject after the candidate reference signal.

The processor may display the signals on the display, based on a Korotkoff sound sensed within an interval based on a peak of an oscillometric signal sensed from the subject.

The processor may determine whether the sound signal is a Korotkoff sound, based on a ratio of frequency components included in a predetermined frequency band among frequency components of the sound signal collected from the subject, and display the signals on the display based on the sound signal determined as the Korotkoff sound.

The processor may determine a sound signal having the ratio greater than or equal to a predetermined threshold ratio to be the Korotkoff sound.

The processor may determine whether the sound signal is a Korotkoff sound according to an amplitude of the sound signal collected from the subject, and display the signals on the display based on the sound signal determined as the Korotkoff sound.

The processor may, after a process of sensing the signals from the subject is completed, even when an amplitude of an ambient signal continuously sensed by the reference signal in an interval based on a peak of an oscillometric signal is less than a predetermined threshold amplitude, in response to a ratio of frequency components included in a predetermined frequency band among frequency components of the ambient signal being greater than a predetermined threshold ratio, change the reference signal to the ambient signal.

When the reference signal corresponds to a diastolic blood pressure (DBP) of the subject, the ambient signal may be a signal sensed at an internal pressure of the cuff that is less than the reference signal, and when the reference signal corresponds to a systolic blood pressure (SBP) of the subject, the ambient signal may be a signal sensed at an internal pressure of the cuff that is greater than the reference signal.

The Korotkoff sound included in the sound signal collected from the subject may be output through a speaker.

A blood pressure measurement result in which the signals and the oscillometric signal sensed from the subject are displayed in synchronization with each other may be provided to the subject, and in the blood pressure measurement result, a visual mark indicative of the reference signal corresponding to a blood pressure of the subject may be displayed, or the reference signal may be visually varied.

The blood pressure measurement result may be output in printed form.

The blood pressure measurement result may be transmitted to an electronic device equipped with a display and displayed on the display of the electronic device.

A blood pressure measuring device according to an embodiment includes a processor configured to detect and display in real time on a display, Korotkoff sounds included in a sound signal collected from a subject based on an oscillometric signal sensed from the subject while an internal pressure of a cuff wrapped around a portion of a body of the subject varies, and visually vary a reference Korotkoff sound or display a visual mark indicative of the reference Korotkoff sound when the reference Korotkoff sound corresponding to a blood pressure of the subject among the Korotkoff sounds is displayed in real time on the display.

The processor may determine whether a candidate reference Korotkoff sound having an amplitude greater than or equal to a predetermined amplitude is detected in an interval based on a peak of the oscillometric signal in the sound signal, and when the candidate reference Korotkoff sound is detected, determine whether the candidate reference Korotkoff sound is a reference Korotkoff sound, based on a ratio of frequency components included in a predetermined frequency band among frequency components of the candidate reference Korotkoff sound.

A blood pressure measuring method according to an embodiment includes sensing a signal from a subject while an internal pressure of a cuff wrapped around a portion of a body of the subject varies, and displaying the signal in real time on a display, wherein the displaying may include visually varying a reference signal or displaying a visual mark indicative of the reference signal when the reference signal corresponding to a blood pressure of the subject among the signals sensed from the subject is displayed in real time on the display.

The displaying may include displaying the signals on the display based on a Korotkoff sound included in a sound signal collected from a microphone while the internal pressure of the cuff varies.

The sensing may include, when a signal that is not sensed from the subject is sensed while the internal pressure of the cuff varies, selecting the signal as a candidate reference signal, and determining whether to determine the candidate reference signal to be the reference signal based on whether a new signal is sensed from the subject after the candidate reference signal.

The sensing may include determining the candidate reference signal to be the reference signal in response to n (where n is a natural number) new signals being continuously sensed from the subject after the candidate reference signal.

The sensing may include sensing the signal, based on a Korotkoff sound sensed within an interval based on a peak of an oscillometric signal sensed from the subject.

The sensing may include determining whether the sound signal is a Korotkoff sound, based on a ratio of frequency components included in a predetermined frequency band among frequency components of the sound signal collected from the subject, and sensing the signal based on the sound signal determined as the Korotkoff sound.

The sensing may include determining a sound signal having the ratio greater than or equal to a predetermined threshold ratio to be the Korotkoff sound.

The sensing may include determining whether the sound signal is a Korotkoff sound according to an amplitude of the sound signal collected from the subject, and sensing the signal based on the sound signal determined as the Korotkoff sound.

A blood pressure measuring device according to an embodiment includes a cuff configured to sense a Korotkoff sound and an oscillometric signal together from a subject, and a processor configured to measure a blood pressure of the subject using the Korotkoff sound and the oscillometric signal.

The processor may sense the Korotkoff sound using the oscillometric signal, and measure the blood pressure of the subject based on the sensed Korotkoff sound.

The Korotkoff sound collected from the subject may be output through a speaker.

A blood pressure measurement result in which the signals and the oscillometric signal sensed from the subject are displayed in synchronization with each other may be provided to the subject, and in the blood pressure measurement result, a visual mark indicative of a reference Korotkoff sound corresponding to the blood pressure of the subject may be displayed, or the reference Korotkoff sound may be visually varied.

The blood pressure measurement result may be output in printed form.

### Effects Of The Invention

According to an embodiment, an oscillometric signal may be used to improve the accuracy of blood pressure measurement based on a Korotkoff sound, thereby providing a blood pressure measuring device and method that are robust to noise or examiner skill.

According to an embodiment, a blood pressure measuring device and method that combines the advantages of auscultation based on a Korotkoff sound and oscillometry based on an oscillometric signal may be provided.

According to an embodiment, when a Korotkoff sound detected while a cuff is being depressurized is displayed in real time on a display, information on systolic blood pressure and diastolic blood pressure may also be displayed in real time on the display, thereby effectively improving a reliability of a subject 150 with respect to a measured blood pressure. By visually verifying the Korotkoff sound detected in real time and visually verifying the systolic blood pressure corresponding to a start point of the Korotkoff sound and the diastolic blood pressure corresponding to an end point of the Korotkoff sound, a reliability of the subject with respect to a measurement result of the blood pressure measuring device may be improved.

According to an embodiment, by providing a subject with visual feedback and/or auditory feedback on a Korotkoff sound sensed during a blood pressure measurement process, a reliability of the subject with respect to a blood pressure measurement result may be effectively improved.

According to an embodiment, a blood pressure measurement result may be provided in printed form.

According to an embodiment, by simultaneously sensing an oscillometric signal and a Korotkoff sound from one cuff, the accuracy and reliability of a blood pressure measurement result may be effectively improved.

### BRIEF DESCRIPTION OF DRAWINGS

FIG. 1 is a diagram illustrating a blood pressure measuring device according to an embodiment.

FIG. 2 is a diagram illustrating an operation of sensing a Korotkoff sound around a peak of an oscillometric signal according to an embodiment.

FIG. 3 is a diagram illustrating an operation of sensing a Korotkoff sound through frequency analysis of a sound signal according to an embodiment.

FIGS. 4 to 7 are diagrams illustrating an operation of determining blood pressure information of a subject using n Korotkoff sounds according to an embodiment.

FIG. 8 is a diagram illustrating an operation of correcting blood pressure information of a subject after blood pressure measurement is completed according to an embodiment.

FIG. 9 is a diagram illustrating a blood pressure measuring method according to an embodiment.

FIG. 10 is a diagram illustrating a blood pressure measuring device according to an embodiment.

FIG. 11 is a diagram illustrating a result printout according to an embodiment.

### BEST MODE FOR CARRYING OUT THE INVENTION

The following detailed structural or functional description is provided as an example only and various alterations and modifications may be made to the embodiments. Accordingly, the embodiments are not construed as limited to the disclosure and should be understood to include all changes, equivalents, and replacements within the idea and the technical scope of the disclosure.

Although terms of “first” or “second” are used to explain various components, the components are not limited to the terms. These terms should be used only to distinguish one component from another component. For example, a first component may be referred to as a second component, and similarly, the second component may also be referred to as the first component.

It will be understood that when a component is referred to as being “connected to” or “coupled” to another component, the component may be directly connected or coupled to the other component or intervening components may be present.

As used herein, the singular forms “a,” “an,” and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Hereinafter, embodiments will be described in detail with reference to the accompanying drawings. When describing the embodiments with reference to the

accompanying drawings, like reference numerals refer to like elements and any repeated description related thereto will be omitted.

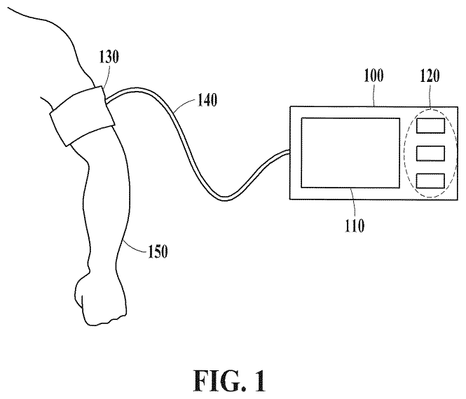

FIG. 1 is a diagram illustrating a blood pressure measuring device according to an embodiment.

A method of measuring blood pressure may include a direct measurement scheme and an indirect measurement scheme. A direct measurement scheme may directly measure intravascular pressure by inserting a catheter into an artery. This scheme may be difficult to use universally because it may cause damage to the vascular system as it requires cannulation of a subject's artery.

A number of studies have been conducted on indirect measurement schemes. Among the indirect measurement schemes, auscultation may be a scheme of measuring blood pressure using a stethoscope, a pressure gauge, and a cuff, and may be the most traditional and standard approach. Auscultation may determine blood pressure based on a

Korotkoff sound generated when a cuff is pressurized and then depressurized. For example, a point at which a Korotkoff sound begins to occur during cuff depressurization may be determined as a systolic blood pressure (SBP), and a point at which a Korotkoff sound no longer occurs and disappears may be determined as a diastolic blood pressure (DBP). However, since auscultation requires an examiner (e.g., medical staff such as a doctor or nurse) to directly listen to a Korotkoff sound generated by pressurizing and then depressurizing a cuff and determine a blood pressure value, the examiner's skill level may have a significant impact on the accuracy of the measurement.

Another indirect measurement scheme, oscillometry, which is relatively easy to measure and is robust to noise, is mainly used in automatic blood pressure monitors. Oscillometry may be a scheme in which pressure is applied to an airbag inside a cuff and then the pressure is gradually reduced, arterial pressure generated by contraction of the heart is transmitted to an upper arm, and a magnitude of the arterial pressure transmitted to the airbag of the cuff wrapped around the upper arm is detected so that the magnitude of the arterial pressure corresponds to an internal pressure of the airbag inside the cuff, and a pressure value inside the airbag at a position where a predetermined magnitude of arterial pressure is located relative to the largest arterial pressure is calculated as a maximum blood pressure and a minimum blood pressure. Oscillometry is widely used in automatic blood pressure monitors due to convenience as the method does not require skilled techniques compared to auscultation and is less susceptible to external noise or movement, making it more stable. However, since there is no clear standard for measuring blood pressure, each blood pressure monitor uses a different blood pressure measurement algorithm, which may cause errors between blood pressure monitors and reduce accuracy.

In order to combine the accuracy of auscultation with the convenience of an oscillometric-based automatic blood pressure monitor, a microphone may be mounted on the cuff of the automatic blood pressure monitor so that Korotkoff sounds sensed by the microphone may be collected together with an oscillometric signal sensed by a pressure sensor that measures pressure changes within the cuff. By using an oscillometric signal, the start and end points of detecting Korotkoff sounds, which are audible signals in the form of impulses that occur when a blood pressure rises above the internal pressure of the cuff, may be identified, and blood pressure information of a subject may be determined based on the start and end points. The present disclosure relates to a blood pressure measuring device and method that are robust to noise and examiner skill by improving the accuracy of blood pressure measurement based on Korotkoff sounds using oscillometric signals.

Referring to FIG. 1, a blood pressure measuring device 100 may include a processor (not shown), a display 110, and an inputter 120.

The processor may control an overall operation of the blood pressure measuring device 100 and may control other components included in the blood pressure measuring device 100.

The display 110 may display a blood pressure measurement process and result. For example, the display 110 may display signals sensed from a subject 150 in real time while an internal pressure of a cuff 130 wrapped around a portion of a body of the subject 150 varies. The display 110 may display blood pressure information of the subject 150 when blood pressure measurement is completed. In addition, the display 110 may display various pieces of information related to the blood pressure measurement without limitation.

The inputter 120 may receive an input related to blood pressure measurement from the subject 150 and/or an examiner. For example, the inputter 120 may receive inputs of the blood pressure measuring device 100 such as start of operation, emergency stop, output of measured blood pressure information or previously stored blood pressure information, but is not limited to the examples described above. In FIG. 1, the inputter 120 is illustrated as a physical button for ease of description, but is not limited thereto, and may be implemented in various forms (e.g., touch, jog dial, switch, and the like). Additionally, according to another embodiment, the inputter 120 may be omitted, and an input may be received from the subject 150 and/or the examiner through the display 110 implemented as a touch screen.

The cuff 130 may include an airbag in which internal pressure is adjusted by injecting fluid or discharging the injected fluid, and the airbag may wrap around a portion of the body of the subject 150 when measuring blood pressure. For example, the portion of the body of the subject 150 may be the upper arm, but is not limited to the above-described example. In the present disclosure, the internal pressure of the airbag in the cuff 130 may also be expressed as the internal pressure of the cuff 130 for ease of description.

A connecting line 140 may include a wire and a fluid tube connecting the blood pressure measuring device 100 and the cuff 130. The wire may electrically connect a sensor (e.g., a microphone, pressure sensor, or the like) within the cuff 130 to the blood pressure measuring device 100. Depending on the embodiment, all or a part of the sensor may be placed within the blood pressure measuring device 100, and a signal (such as an oscillometric signal and/or Korotkoff sound) obtained from the cuff 130 may be transmitted to the sensor within the blood pressure measuring device 100 via the connecting line 140. The fluid tube may deliver fluid to be injected into the airbag within the cuff 130 or fluid discharged from the airbag.

The blood pressure measuring device 100 may collect, using a microphone, a sound signal generated when the cuff 130 is pressurized to a level higher than a normal SBP and then slowly depressurized while the cuff 130 is wrapped around the upper arm of the subject 150, and may sense an oscillometric signal from a pressure sensor. The blood pressure measuring device 100 may detect a Korotkoff sound from the sound signal by analyzing the sound signal and the oscillometric signal, and may determine an SBP and DBP of the subject 150 in real time based on the detected Korotkoff sound. In addition, the blood pressure measuring device 100 may display the detected Korotkoff sound in real time on the display 110, and when a reference Korotkoff sound corresponding to the SBP and a reference Korotkoff sound corresponding to the DBP are displayed in real time on the display 110, the reference Korotkoff sound may be visually varied (e.g., by different colors, different line thicknesses, different line shapes, and the like), or a visual mark (e.g., an arrow, triangle, or the like) indicative of the reference Korotkoff sound may be displayed.

When the subject 150 measures the blood pressure by wrapping the cuff 130 around the upper arm without any additional equipment, the blood pressure measuring device 100 may sense both the oscillometric signal and the Korotkoff sound from the subject 150 through the cuff 130. A sound receiver (e.g., a microphone) for sensing a Korotkoff sound may be placed and connected to the cuff 130 so that the sound receiver fits closely to the body of the subject 150 wearing the cuff 130. The pressure sensor for detecting an oscillometric signal may also be placed within the cuff 130, or a device and/or component for transmitting a signal to the pressure sensor placed within the blood pressure measuring device 100 may be placed within the cuff 130.

When the Korotkoff sound detected while the cuff 130 is depressurized is displayed in real time on the display 110, information on the SBP and DBP may also be displayed in real time on the display 110, thereby effectively improving a reliability of the subject 150 with respect to a measured blood pressure. The subject 150 may visually verify a Korotkoff sound detected in real time, and visually verify an SBP corresponding to a start point of the Korotkoff sound and a DBP corresponding to an end point of the Korotkoff sound, thereby improving the reliability of the subject 150 with respect to a measurement result of the blood pressure measuring device 100.

Additionally, the Korotkoff sound detected while the cuff 130 is depressurized may be output through a speaker (not shown). The reliability of a blood pressure measurement result may be improved by providing the subject 150 with real-time auditory feedback of the detected Korotkoff sound. As described above, the subject 150 may effectively improve satisfaction with a blood pressure measurement result by directly verifying the blood pressure measurement result through hearing and sight by receiving real-time visual feedback of the Korotkoff sound detected through the display 110. A speaker that outputs a Korotkoff sound may be built into the blood pressure measuring device 100, but is not limited thereto, and the speaker may be a separate device that receives a Korotkoff sound detected from the blood pressure measuring device 100 via a wired and/or wireless network. Hereinafter, for ease of description, an example of a case where a detected Korotkoff sound is displayed on the display 110 is described, but the description may also be applied to a case where the Korotkoff sound is output through a speaker.

According to an embodiment, the blood pressure measuring device 100 may combine the advantages of auscultation and oscillometry by using the characteristics (e.g., a correlation between Korotkoff sounds and oscillometric signals, etc.) of Korotkoff sounds and oscillometric signals. The blood pressure measuring device 100 may sense an oscillometric signal. The blood pressure measuring device 100 may determine whether a Korotkoff sound is detected in an interval based on a peak of an oscillometric signal and whether an amplitude of the Korotkoff sound is greater than or equal to a predetermined threshold amplitude. When a Korotkoff sound greater than or equal to a predetermined threshold amplitude is detected in an interval based on the peak of the oscillometric signal, the blood pressure measuring device 100 may determine whether the detected Korotkoff sound is noise through frequency analysis of the Korotkoff sound. When it is determined that the detected Korotkoff sound is not noise, the blood pressure measuring device 100 may display the corresponding Korotkoff sound in real time on the display 110.

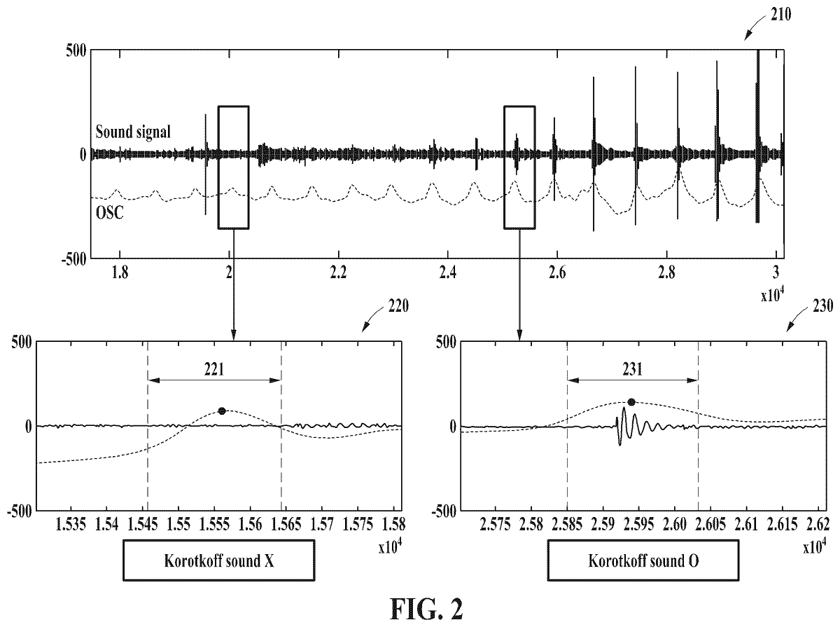

FIG. 2 is a diagram illustrating an operation of sensing a Korotkoff sound around a peak of an oscillometric signal according to an embodiment.

Referring to FIG. 2, an example is illustrated to describe an operation of detecting a Korotkoff sound from a sound signal by using a correlation between a Korotkoff sound and an oscillometric signal.

Variation in blood pressure due to heartbeats may appear in the form of pulses, and these pulse forms may also be reflected in an oscillometric signal. Additionally, since a Korotkoff sound occurs at a moment when the blood pressure rises above an internal pressure of a cuff, the Korotkoff sound may appear corresponding to a peak in the oscillometric signal. By using such characteristic, a Korotkoff sound may be detected with high accuracy near the peak of the oscillometric signal. In general, the peak of an oscillometric signal is more robust to noise and easier to detect than the Korotkoff sound, so the accuracy of Korotkoff sound detection may be improved by first detecting the peak of the oscillometric signal and then detecting the Korotkoff sound near the peak.

The blood pressure measuring device may sense a Korotkoff sound within an interval based on a peak of an oscillometric signal sensed from a subject.

A screen 210 displayed on a display of the blood pressure measuring device may include a sound signal collected from a microphone and an oscillometric signal sensed from a pressure sensor (indicated as OSC in FIG. 2).

A first case 220 may represent an example in which the internal pressure of the cuff is higher than the blood pressure and thus a Korotkoff sound does not occur. The blood pressure measuring device may determine whether the sound signal contains a Korotkoff sound within an interval 221 based on the peak of the oscillometric signal. The interval 221 may be set to have a predetermined magnitude based on the peak of the oscillometric signal, but is not limited thereto, and for example, the interval 221 may be set to have a dynamic magnitude depending on the magnitude of the peak. Since the sound signal remains relatively constant in the interval 221, the blood pressure measuring device may determine that there is no Korotkoff sound in the interval 221.

A second case 230 may represent an example in which a Korotkoff sound occurs because the blood pressure is higher than the internal pressure of a cuff. The blood pressure measuring device may determine whether the sound signal contains a Korotkoff sound within an interval 231 based on the peak of the oscillometric signal. The blood pressure measuring device may detect an impulse-shaped wave included in the sound signal as a Korotkoff sound in the corresponding interval 221.

The blood pressure measuring device may detect a Korotkoff sound in a portion of a sound signal rather than the entire sound signal based on a peak of an oscillometric signal, thereby minimizing resources required for Korotkoff sound detection and improving the accuracy of Korotkoff sound detection.

Additionally, the blood pressure measuring device may determine whether a sound signal is a Korotkoff sound based on an amplitude of the sound signal collected from the subject. Since an audible signal that may actually be heard by the human ear in a sound signal is the Korotkoff sound, the Korotkoff sound may have an amplitude greater than a predetermined reference. Therefore, the blood pressure measuring device may determine whether a sound signal is a Korotkoff sound based on the amplitude of the sound signal. For example, the amplitude of a sound signal may be determined by a maximum difference between a baseline and the corresponding signal, or a difference between the maximum and minimum of the corresponding signal. The blood pressure measuring device may determine whether the sound signal is a Korotkoff sound based on whether the amplitude of the sound signal determined in this manner is greater than a predetermined threshold amplitude.

Additionally, the blood pressure measuring device may determine whether the sound signal is a Korotkoff sound based on whether the sound signal is audible. For example, a sound that a human may hear may be expressed in dbSPL values (or decibels), and a sound that a human may hear starts at 0 decibels, but an average person may hear from around 20 decibels. Also, humans may have difficulty hearing low-frequency sounds and may hear high-frequency sounds better even at the same loudness. The relationship between a human's hearing level and frequency may also be used to determine whether a signal is audible.

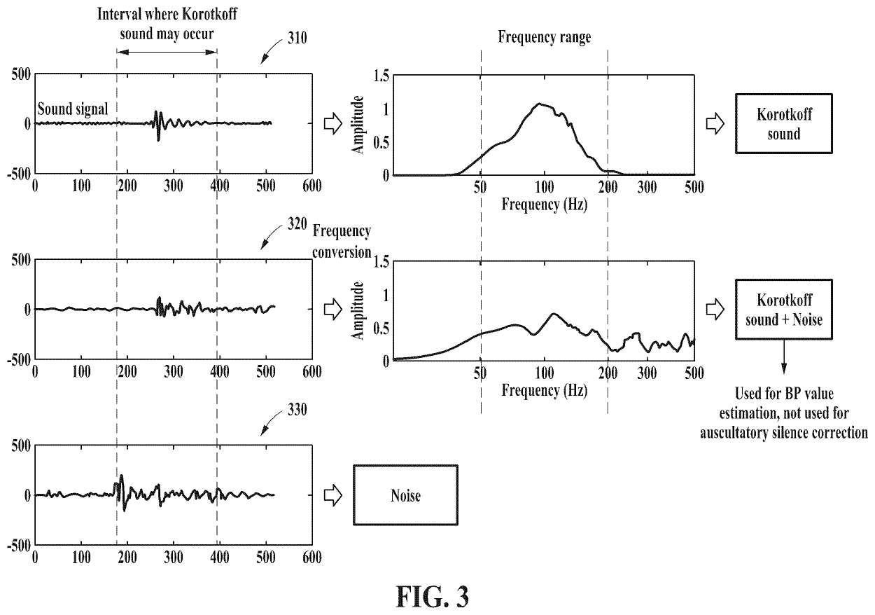

FIG. 3 is a diagram illustrating an operation of sensing a Korotkoff sound through frequency analysis of a sound signal according to an embodiment.

Referring to FIG. 3, an example is illustrated to describe an operation of determining whether a sound signal contains a Korotkoff sound and noise through frequency analysis.

Since a Korotkoff sound is an audible signal, the Korotkoff sound may have a characteristic of typically existing in a particular frequency band (e.g., 50-200 Hz). The blood pressure measuring device may use the frequency band characteristic of a Korotkoff sound to determine whether a sound signal is a Korotkoff sound or whether the sound signal contains noise. Frequency analysis may be performed on a sound signal within a range where a Korotkoff sound may occur. For example, the frequency analysis may be performed on a sound signal first detected as a Korotkoff sound in FIG. 2.

The blood pressure measuring device may determine whether a sound signal is a Korotkoff sound based on a ratio of frequency components included in a predetermined frequency band (e.g., 50-200 Hz) among frequency components of a sound signal collected from a subject.

For example, in a first case 310 in which a ratio between the frequency components included in a predetermined frequency band and frequency components included in the remaining frequency band is greater than or equal to a predetermined first threshold ratio, the blood pressure measuring device may determine the sound signal as a Korotkoff sound.

Alternatively, in a second case 320 in which a ratio between the frequency components included in the predetermined frequency band and the frequency components included in the remaining frequency band is less than the predetermined first threshold ratio and greater than or equal to a predetermined second threshold ratio, the blood pressure measuring device may determine the sound signal as a Korotkoff sound containing noise. In this example, the second threshold ratio may be set lower than the first threshold ratio. Because the Korotkoff sound contains noise, the blood pressure measuring device may use the Korotkoff sound to estimate blood pressure values but may not use it for auscultatory silence correction.

Alternatively, in a third case 330 in which a ratio between the frequency components included in the predetermined frequency band and the frequency components included in the remaining frequency band is less than the predetermined second threshold ratio, the blood pressure measuring device may determine the sound signal as noise.

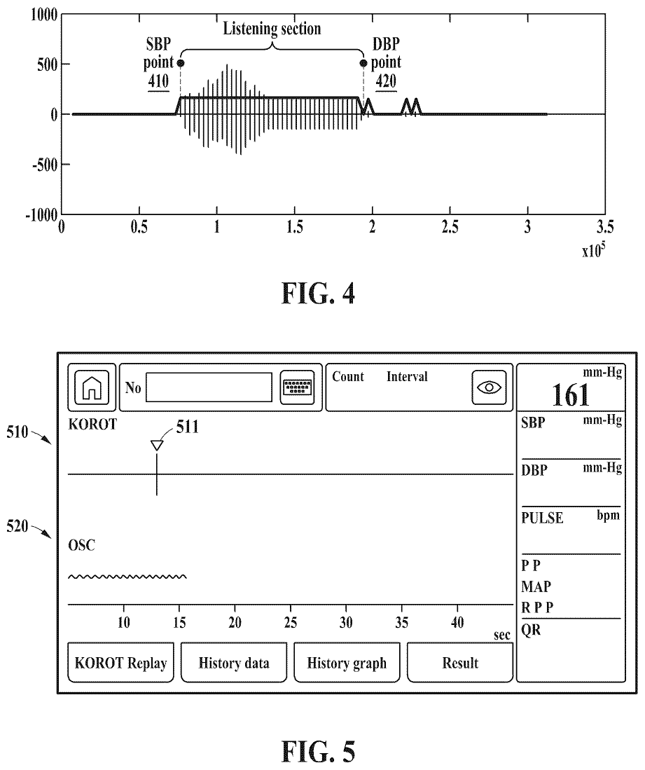

FIGS. 4 to 7 are diagrams illustrating an operation of determining blood pressure information of a subject using n Korotkoff sounds according to an embodiment.

Referring to FIG. 4, since a Korotkoff sound occurs when blood pressure is higher than an internal pressure of the cuff, the Korotkoff sound may have a characteristic of occurring continuously in accordance with a heartbeat cycle from an SBP point 410 to a DBP point 420, and the blood pressure measuring device may correct incorrectly detected Korotkoff sounds in real time by using this characteristic of Korotkoff sounds.

Even when the internal pressure of the cuff is greater than the blood pressure, a temporary impulse-type sound signal may be collected. In some cases, when an impulse-type sound signal satisfies the reference described in FIGS. 2 and 3, the blood pressure measuring device may misrecognize the corresponding sound signal as a Korotkoff sound. However, as described above, while a normal Korotkoff sound may have the characteristic of occurring continuously in accordance with the heartbeat cycle from the SBP point 410 to the DBP point 420, no subsequent Korotkoff sound may be detected after a temporary impulse-type sound signal.

The blood pressure measuring device may select a signal as a candidate reference Korotkoff sound when a Korotkoff sound that was not sensed from the subject is sensed while the internal pressure of the cuff varies, and may determine whether to determine the candidate reference Korotkoff sound as a reference Korotkoff sound corresponding to an SBP based on whether a new Korotkoff sound is sensed from the subject after the candidate reference Korotkoff sound. The blood pressure measuring device may determine the candidate reference Korotkoff sound as the reference Korotkoff sound in response to n (where n is a natural number) new Korotkoff sounds being sensed continuously from the subject after the candidate reference Korotkoff sound.

Referring to FIG. 5, an example is shown in which a Korotkoff sound is temporarily sensed and selected as a candidate reference Korotkoff sound 511, and no new Korotkoff sound is sensed after the candidate reference Korotkoff sound 511. A screen displayed on a display may show a Korotkoff sound 510 and an oscillometric signal 520. The oscillometric signal 520 may be a direct display of a value sensed by a pressure sensor, but the Korotkoff sound 510 may only display a value detected as a Korotkoff sound from a sound signal collected by a microphone. While an internal pressure of the cuff varies over time, the oscillometric signal 520 is continuously sensed, when the candidate reference Korotkoff sound 511 is sensed once and then a subsequent Korotkoff sound is not sensed, the candidate reference Korotkoff sound 511 may not be ultimately selected as the reference Korotkoff sound.

Referring to FIG. 6, an example is shown in which a Korotkoff sound is temporarily sensed and selected as a candidate reference Korotkoff sound 610, and a new Korotkoff sound is sensed after the candidate reference Korotkoff sound 610. Since n new Korotkoff sounds are sensed continuously after the candidate reference Korotkoff sound 610, the blood pressure measuring device may determine the candidate reference Korotkoff sound 610 as a reference Korotkoff sound corresponding to an SBP, and may visually vary the reference Korotkoff sound from other Korotkoff sounds, or display a visual mark (e.g., a triangle, etc.) indicative of the reference Korotkoff sound. In FIG. 6, for ease of description, an example in which n=4 is illustrated, but n may also have a natural number value.

Referring to FIG. 7, an example is illustrated in which visual marks (e.g., triangles, etc.) indicative of a reference Korotkoff sound 710 corresponding to an SBP and a reference Korotkoff sound 720 corresponding to a DBP are displayed according to the method described above. In FIG. 7, visual marks indicating the reference Korotkoff sounds 710 and 720 are shown for ease of description, but the embodiment is not limited thereto, and the reference Korotkoff sounds 710 and 720 may be indicated differently from other Korotkoff sounds.

The blood pressure measuring device may sense Korotkoff sounds continuously and, when Korotkoff sounds are no longer sensed, determine a last sensed Korotkoff sound as the reference Korotkoff sound 720 corresponding to the DBP. Once blood pressure measurement is completed, the display screen may display not only the sensed Korotkoff sounds and oscillometric signals, but also various pieces of blood pressure-related information (e.g., SBP, DBP, pulse, and the like).

The Korotkoff sounds shown in FIGS. 5 to 7 may be detected according to the Korotkoff sound detection scheme described above.

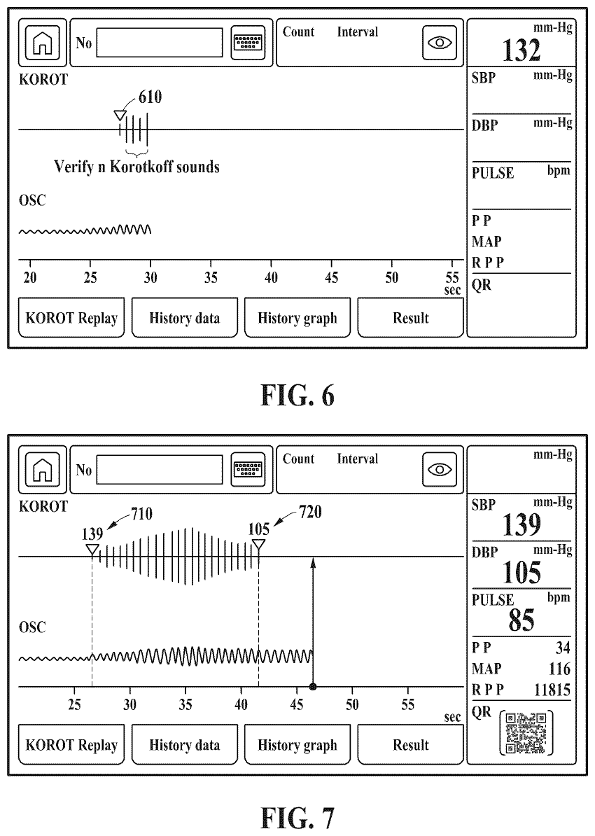

FIG. 8 is a diagram illustrating an operation of correcting blood pressure information of a subject after blood pressure measurement is completed, according to an embodiment.

Referring to FIG. 8, an example is shown in which a reference Korotkoff sound 810 corresponding to a DBP is corrected to an ambient Korotkoff sound 820 after the blood pressure measurement is completed.

The blood pressure measuring device may variably adjust a reference used to detect Korotkoff sounds by analyzing characteristics of a sound signal being measured. Even when an amplitude of a sound signal collected from a microphone after a reference Korotkoff sound corresponding to a DBP is small, when a Korotkoff sound is detected around a peak of an oscillometric signal and a ratio of frequency components included in a predetermined frequency band among frequency components of the corresponding Korotkoff sound is greater than or equal to a predetermined threshold ratio, the blood pressure measuring device may change the reference Korotkoff sound 810 to the ambient Korotkoff sound 820. For example, the blood pressure measuring device may change the reference Korotkoff sound 810 to the ambient Korotkoff sound 820 a predetermined number of times.

After blood pressure measurement is completed, by correcting the reference Korotkoff sound 810 to the ambient Korotkoff sound 820, the reference Korotkoff sound may be detected more accurately even when the subject's Korotkoff sound itself is small.

In FIG. 8, for ease of description, an example is shown in which the reference Korotkoff sound 810 corresponding to the DBP is corrected to a subsequently measured ambient Korotkoff sound 820, but the above description may also be applied to an example in which a reference Korotkoff sound corresponding to an SBP is corrected to a previously measured ambient Korotkoff sound.

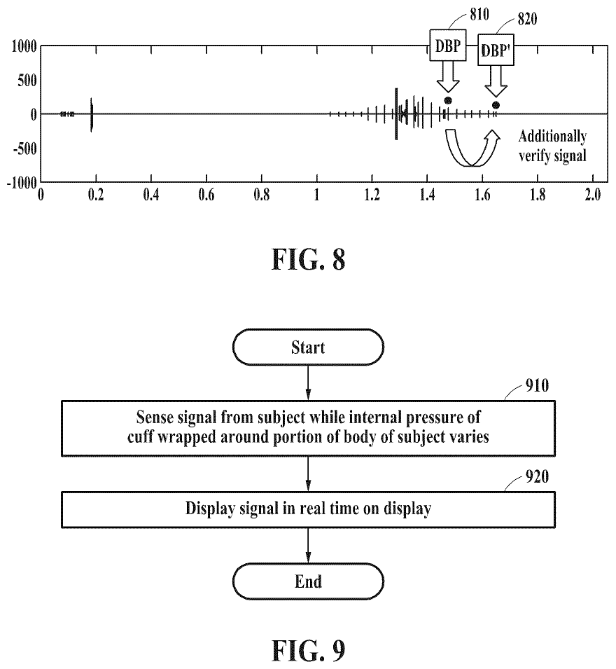

FIG. 9 is a diagram illustrating a blood pressure measuring method according to an embodiment.

In the following embodiments, operations may be performed sequentially, but not necessarily performed sequentially. For example, the order of the operations may change, and at least two of the operations may be performed in parallel. Operations 1001 to 1010 may be performed by at least one component (e.g., a processor, a sensor, etc.) of an electronic device.

In the following embodiments, operations may be performed sequentially, but not necessarily performed sequentially. For example, the order of the operations may change, and at least two of the operations may be performed in parallel. Operations 910 and 620 may be performed by at least one component (e.g., a processor, etc.) of a blood pressure measuring device.

In operation 910, the blood pressure measuring device senses a signal from a subject while an internal pressure of a cuff wrapped around a portion of a body of the subject varies.

The blood pressure measuring device may select a signal as a candidate reference signal when a signal that was not sensed from the subject is sensed while the internal pressure of the cuff varies, and may determine whether to determine the candidate reference signal as a reference signal based on whether a new signal is sensed from the subject after the candidate reference signal. The blood pressure measuring device may determine the candidate reference signal as the reference signal in response to n (where n is a natural number) new signals being sensed continuously from the subject after the candidate reference signal.

The blood pressure measuring device may sense a signal based on a Korotkoff sound sensed within an interval based on a peak of an oscillometric signal sensed from the subject.

The blood pressure measuring device may determine whether a sound signal is a Korotkoff sound based on a ratio of frequency components included in a predetermined frequency band among frequency components of a sound signal collected from the subject, and sense a signal based on the sound signal determined as the Korotkoff sound. The blood pressure measuring device may determine a sound signal having a ratio greater than or equal to a predetermined threshold ratio as a Korotkoff sound.

The blood pressure measuring device may determine whether a sound signal is a Korotkoff sound based on an amplitude of the sound signal collected from the subject, and sense the signal based on the sound signal determined as a Korotkoff sound.

In operation 920, the blood pressure measuring device displays the signal in real time on the display. When a reference signal corresponding to the blood pressure of a subject among the signals sensed from the subject is displayed in real time on the display, the blood pressure measuring device may visually vary the reference signal or display a visual mark indicative of the reference signal. The blood pressure measuring device may display a signal on the display based on Korotkoff sounds included in a sound signal collected by a microphone while the internal pressure of the cuff varies.

The descriptions provided with reference to FIGS. 1 to 8 may apply to the operations shown in FIG. 9, and thus further detailed descriptions are omitted.

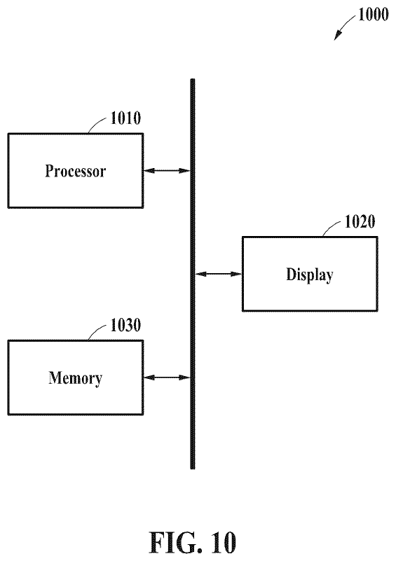

FIG. 10 is a diagram illustrating a blood pressure measuring device according to an embodiment.

Referring to FIG. 10, a blood pressure measuring device 1000 includes a processor 1010. Additionally, the blood pressure measuring device 1000 may further include a display 1020 and a memory 1030. The processor 10140, the display 1020, and the memory 1030 may communicate with each other via a bus, a Peripheral Component Interconnect Express (PCIe), and/or a Network on a Chip (NoC).

The processor 1010 displays signals sensed from a subject in real time on a display while an internal pressure of a cuff wrapped around a portion of the subject's body varies, and when a reference signal corresponding to the subject's blood pressure among the signals is displayed in real time on the display, the reference signal is visually varied or a visual mark indicative of the reference signal is displayed.

The processor 1010 may display the signals on the display based on a Korotkoff sound included in a sound signal collected from a microphone while the internal pressure of the cuff varies.

The processor 1010 may select a signal as a candidate reference signal when a

signal that was not sensed from the subject is sensed while the internal pressure of the cuff varies, and may determine whether to determine the candidate reference signal as a reference signal based on whether a new signal is sensed from the subject after the candidate reference signal. The processor 1010 may determine the candidate reference signal as the reference signal in response to n (where n is a natural number) new signals being sensed continuously from the subject after the candidate reference signal.

The processor 1010 may display signals on the display based on a Korotkoff sound sensed within an interval based on a peak of an oscillometric signal sensed from the subject.

The processor 1010 may determine whether a sound signal is a Korotkoff sound based on a ratio of frequency components included in a predetermined frequency band among frequency components of a sound signal collected from the subject, and display signals on the display based on the sound signal determined as the Korotkoff sound. The processor 1010 may determine a sound signal having a ratio greater than or equal to a predetermined threshold ratio as a Korotkoff sound.

The processor 1010 may determine whether a sound signal is a Korotkoff sound based on an amplitude of the sound signal collected from the subject, and display signals on the display based on the sound signal determined as a Korotkoff sound.

After the process of sensing signals from the subject is completed, the processor 1010 may change the reference signal to an ambient signal when, in an interval based on a peak of an oscillometric signal, even when an amplitude of the ambient signal sensed continuously based on the reference signal is less than a predetermined threshold amplitude, a ratio of frequency components included in a predetermined frequency band among frequency components of the ambient signal is greater than or equal to a predetermined threshold ratio. When the reference signal corresponds to a DBP of the subject, the ambient signal may be a signal sensed at an internal pressure of a cuff that is lower than the reference signal, and when the reference signal corresponds to an SBP of the subject, the ambient signal may be a signal sensed at an internal pressure of the cuff that is higher than the reference signal.

In addition, the processor 1010 detects Korotkoff sounds included in a sound signal collected from the subject based on an oscillometric signal sensed from the subject while the internal pressure of the cuff wrapped around a portion of the subject's body varies, and displays the detected Korotkoff sounds in real time on a display, and when a reference Korotkoff sound corresponding to the subject's blood pressure among the Korotkoff sounds is displayed in real time on the display, the reference Korotkoff sound is visually varied or a visual mark indicative of the reference Korotkoff sound is displayed. The processor 1010 determines whether a candidate Korotkoff sound having an amplitude greater than or equal to a predetermined amplitude is detected in an interval based on a peak of an oscillometric signal within a sound signal, and when a candidate Korotkoff sound is detected, the processor 1010 may determine whether the candidate Korotkoff sound is a Korotkoff sound based on a ratio of frequency components included in a predetermined frequency band among frequency components of the candidate Korotkoff sound.

The display 1020 may display signals sensed from the subject in real time while the internal pressure of the cuff wrapped around a portion of the subject's body varies, based on the control of the processor 1010. When a reference signal corresponding to the blood pressure of the subject among the signals is displayed in real time on the display, the display 1020 may visually vary the reference signal or display a visual mark indicative of the reference signal.

The memory 1030 may include computer-readable instructions. The processor 1010 may perform the operations described above when the instructions stored in the memory 1030 are executed by the processor 1010. The memory 1030 may be a volatile memory or a non-volatile memory.

In addition, the blood pressure measuring device 1000 may process the operations described above.

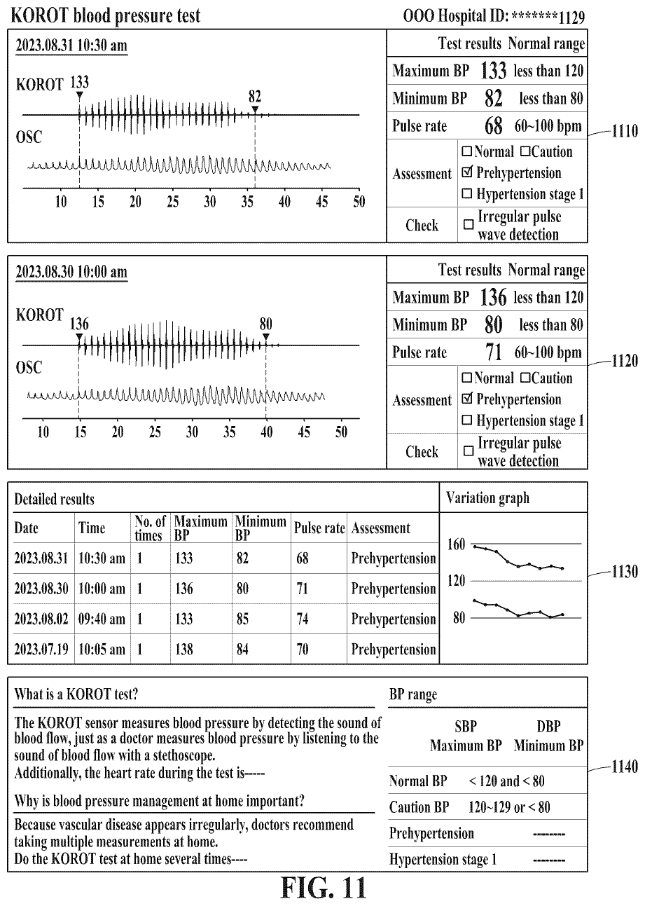

FIG. 11 is a diagram illustrating a result printout according to an embodiment.

Referring to FIG. 11, an example of a result printout provided when the blood pressure measurement of a subject is completed is illustrated. The result printout may be provided to a subject and/or medical staff in printed form. For example, the printout may have an A4 paper size or letter paper size, but is not limited thereto. In FIG. 11, for ease of description, an example is shown in which the blood pressure measurement result is provided in the form of a result printout, but a form in which the blood pressure measurement result is provided is not limited to the above-described example, and the blood pressure measurement result may be displayed on a display provided in a blood pressure measuring device or a display provided in a separate device that receives the blood pressure measurement result from a blood pressure measuring device.

A first item 1110 on the result printout may display the most recent blood pressure measurement result. Korotkoff sounds and oscillometric signals may be displayed in synchronization with each other, and visual marks indicative of an SBP and a DBP of the subject may be displayed together.

A second item 1120 on the result printout may display a specific blood pressure measurement result set by the subject and/or the medical staff. For example, when a bookmark is set for a blood pressure measurement result at an important timepoint or a turning point, the bookmark is displayed in the second item 1120 of the result printout to be easily compared with the most recent blood pressure measurement result in the first item.

A third item 1130 on the result printout may display a history of a previous blood pressure measurement result. Depending on the embodiment, the previous history may be provided in a table and graph form.

A fourth item 1140 on the result printout may display various pieces of information that may be useful to the subject.

The result printout may include a total of four items 1110 to 1140, but is not limited thereto, and may be provided in various forms.

The embodiments described herein may be implemented using a hardware component, a software component and/or a combination thereof. A processing device may be implemented using one or more general-purpose or special-purpose computers, such as, for example, a processor, a controller and an arithmetic logic unit (ALU), a digital signal processor (DSP), a microcomputer, a field programmable gate array (FPGA), a programmable logic unit (PLU), a microprocessor or any other device capable of responding to and executing instructions in a defined manner. The processing device may run an operating system (OS) and one or more software applications that run on the OS. The processing device also may access, store, manipulate, process, and create data in

response to execution of the software. For purpose of simplicity, the description of a processing device is singular; however, one of ordinary skill in the art will appreciate that a processing device may include a plurality of processing elements and a plurality of types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be stored in any type of machine, component, physical or virtual equipment, or computer storage medium or device capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs and DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher-level code that may be executed by the computer using an interpreter.

The above-described hardware devices may be configured to act as one or more software modules in order to perform the operations of the above-described embodiments, or vice versa.

Although the embodiments have been described with reference to the limited drawings, one of ordinary skill in the art may apply various technical modifications and variations based thereon. For example, suitable results may be achieved if the described techniques are performed in a different order, and/or if components in a described system, structure, device, or circuit are combined in a different manner, and/or replaced or supplemented by other components or their equivalents.

Therefore, other implementations, other embodiments, and equivalents to the claims are also within the scope of the following claims.

1. A blood pressure measuring device comprising a processor configured to display signals sensed from a subject in real time on a display while an internal pressure of a cuff wrapped around a portion of a body of the subject varies, and visually vary a reference signal or display a visual mark indicative of the reference signal when the reference signal corresponding to a blood pressure of the subject among the signals is displayed in real time on the display.

2. The blood pressure measuring device of claim 1, wherein the processor is configured to display the signals on the display based on a Korotkoff sound comprised in a sound signal collected from a microphone while the internal pressure of the cuff varies.

3. The blood pressure measuring device of claim 1, wherein the processor is configured to when a signal that is not sensed from the subject is sensed while the internal pressure of the cuff varies, select the signal as a candidate reference signal, and determine whether to determine the candidate reference signal to be the reference signal based on whether a new signal is sensed from the subject after the candidate reference signal.

4. The blood pressure measuring device of claim 3, wherein the processor is configured to determine the candidate reference signal to be the reference signal in response to n (where n is a natural number) new signals being continuously sensed from the subject after the candidate reference signal.

5. The blood pressure measuring device of claim 1, wherein the processor is configured to display the signals on the display, based on a Korotkoff sound sensed within an interval based on a peak of an oscillometric signal sensed from the subject.

6. The blood pressure measuring device of claim 1, wherein the processor is configured to determine whether the sound signal is a Korotkoff sound, based on a ratio of frequency components comprised in a predetermined frequency band among frequency components of the sound signal collected from the subject, and display the signals on the display based on the sound signal determined as the Korotkoff sound.

7. The blood pressure measuring device of claim 6, wherein the processor is configured to determine a sound signal having the ratio greater than or equal to a predetermined threshold ratio to be the Korotkoff sound.

8. The blood pressure measuring device of claim 1, wherein the processor is configured to determine whether the sound signal is a Korotkoff sound according to an amplitude of the sound signal collected from the subject, and display the signals on the display based on the sound signal determined as the Korotkoff sound.

9. The blood pressure measuring device of claim 1, wherein the processor is configured to after a process of sensing the signals from the subject is completed, even when an amplitude of an ambient signal continuously sensed by the reference signal in an interval based on a peak of an oscillometric signal is less than a predetermined threshold amplitude, in response to a ratio of frequency components comprised in a predetermined frequency band among frequency components of the ambient signal being greater than a predetermined threshold ratio, change the reference signal to the ambient signal.

10. The blood pressure measuring device of claim 9, wherein when the reference signal corresponds to a diastolic blood pressure (DBP) of the subject, the ambient signal is a signal sensed at an internal pressure of the cuff that is less than the reference signal, and when the reference signal corresponds to a systolic blood pressure (SBP) of the subject, the ambient signal is a signal sensed at an internal pressure of the cuff that is greater than the reference signal.

11. The blood pressure measuring device of claim 1, wherein the Korotkoff sound comprised in the sound signal collected from the subject is output through a speaker.

12. The blood pressure measuring device of claim 1, wherein a blood pressure measurement result in which the signals and the oscillometric signal sensed from the subject are displayed in synchronization with each other is provided to the subject, and in the blood pressure measurement result, a visual mark indicative of the reference signal corresponding to a blood pressure of the subject is displayed, or the reference signal is visually varied.

13. The blood pressure measuring device of claim 12, wherein the blood pressure measurement result is output in printed form.

14. The blood pressure measuring device of claim 12, wherein the blood pressure measurement result is transmitted to an electronic device equipped with a display and displayed on the display of the electronic device.

15. A blood pressure measuring device comprising a processor configured to detect and display in real time on a display, Korotkoff sounds comprised in a sound signal collected from a subject based on an oscillometric signal sensed from the subject while an internal pressure of a cuff wrapped around a portion of a body of the subject varies, and visually vary a reference Korotkoff sound or display a visual mark indicative of the reference Korotkoff sound when the reference Korotkoff sound corresponding to a blood pressure of the subject among the Korotkoff sounds is displayed in real time on the display.

16. The blood pressure measuring device of claim 15, wherein the processor is configured to determine whether a candidate reference Korotkoff sound having an amplitude greater than or equal to a predetermined amplitude is detected in an interval based on a peak of the oscillometric signal in the sound signal, and when the candidate reference Korotkoff sound is detected, determine whether the candidate reference Korotkoff sound is a reference Korotkoff sound, based on a ratio of frequency components comprised in a predetermined frequency band among frequency components of the candidate reference Korotkoff sound.

17. A blood pressure measuring method, comprising: sensing a signal from a subject while an internal pressure of a cuff wrapped around a portion of a body of the subject varies; and displaying the signal in real time on a display, wherein the displaying comprises visually varying a reference signal or displaying a visual mark indicative of the reference signal when the reference signal corresponding to a blood pressure of the subject among the signals sensed from the subject is displayed in real time on the display.

18-24. (canceled)

25. A blood pressure measuring device, comprising:
a cuff configured to sense a Korotkoff sound and an oscillometric signal together from a subject; and
a processor configured to measure a blood pressure of the subject using the Korotkoff sound and the oscillometric signal.

26. The blood pressure measuring device of claim 25, wherein the processor is configured to sense the Korotkoff sound using the oscillometric signal, and measure the blood pressure of the subject based on the sensed Korotkoff sound.

27. The blood pressure measuring device of claim 25, wherein the Korotkoff sound collected from the subject is output through a speaker.

28. The blood pressure measuring device of claim 25, wherein a blood pressure measurement result in which the signals and the oscillometric signal sensed from the subject are displayed in synchronization with each other is provided to the subject, and in the blood pressure measurement result, a visual mark indicative of a reference Korotkoff sound corresponding to the blood pressure of the subject is displayed, or the reference Korotkoff sound is visually varied.

29. The blood pressure measuring device of claim 28, wherein the blood pressure measurement result is output in printed form.
